(19) United States
(12) Patent Application Publication
(10) Pub. No.: US 2006/0063122 A1
(43) Pub. Date: Mar. 23, 2006

(54) Heating device for samples in the field of life sciences

(76) Inventors: Hubert Heeg, Moembris (DE); Stefan Ferger, Ranstadt (DE); Gerd Ross, Frankfurt (DE)

Correspondence Address: Leo J. Jennings, Esquire;BAKER & HOSTETLER LLP, Washington Square, Suite 1100, 1050 Connecticut Avenue, N.W., Washington, DC, 20036-5304, US

(21) Appl. No.: 11/213,706

(22) Filed: Aug. 30, 2005

(30) Foreign Application Priority Data
Sep. 10, 2004 (DE) 102004043909.5

(51) Int. Cl. F27B 9/04; C12M 1/36; C12M 3/00

(52) U.S. Cl. 432/152; 435/286.6

(57) ABSTRACT

The present invention relates to a heating device for samples in the field of life sciences having at least one cassette with horizontally arranged storage compartments for storing sample carriers in which gas is guided onto the cassette for heating the samples. The present invention also relates to a process for heating such samples that are arranged inside a storage room and in which gas is guided onto the samples for heating them. The heating device comprises a housing with a storage room arranged therein that is designed for the feed and discharge of gases and for the storage of the cassettes. Furthermore, a gas-recirculating device is also provided that is used for recirculating the ambient gas of the storage room. The advantage of this invention is that the samples are heated quickly, evenly, and gently.

The present invention relates to a heating device for samples in the field of life sciences having at least one cassette with horizontally arranged storage compartments for storing sample carriers in which gas is guided onto the cassette for heating the samples. The present invention also relates to a process for heating such samples. The term life sciences generally refers to “the science of life” that deals with the gain and use of scientific knowledge for instance in the fields of chemistry, biology, pharmaceutics, biotechnology, physics and biochemistry. Within the framework of scientific research in this field, it is frequently necessary to heat samples that are usually stored in sample carriers, particularly in microtiter plates. It is often desirable to heat the samples at a definite temperature. A typical example of application is the thawing of frozen samples.

Usually the sample carriers are stored in cassettes that comprise horizontally arranged storage compartments for accommodating the sample carriers. The storage compartments are normally arranged above one another and every compartment comprises at least one opening for the insertion of the sample carriers. Furthermore, every compartment comprises at least one opening so that the gas surrounding the cassettes can flow through the compartments.

It is known that samples are exposed to the ambient temperature for thawing purposes or that heated air from the ambience is guided onto the cassettes equipped with the samples. However, in doing so, it is not always possible to ensure an even temperature pattern for all the samples.

The objective of the present invention is to specify a heating device as well as a process for heating samples in which the samples can be heated in a controlled and even manner.

This objective is achieved in the heating device of the type described above by the heating device that comprises of a housing with a storage room arranged therein designed for the feed and discharge of gas and for storing the cassette. A gas-recirculating device is provided in the storage room for recirculating the gas.

Due to the presence of a housing using which the storage room can be locked from the ambience, the samples stored in the storage room are not exposed to the ambience, thus reducing the risk of contamination. The samples can be inserted into the storage room either manually or using an automatic loading and unloading system. Such an automatic loading and unloading device is known for instance from the patent application U.S. Pat. No. 6,129,428. After the samples are stored into the storage room, the housing is sealed and the storage room is thus sealed off from the ambience.

An additional advantage of the present invention is that the design of the storage room for the feed and discharge of gas makes it possible for guiding the gas onto the cassettes for heating the samples. The closed arrangement of the storage room enables the control of the gas flow and thus enables the samples to be heated evenly. Moreover, due to the gas-recirculating device it is possible to use only the gas that is already present in the storage room for heating the samples without the risk of a contamination from the ambient air. Using the present invention it is possible to execute the heating process, particularly the thawing process of the samples with controlled speed, evenly and gently so as to enable the samples to be processed further without them being contaminated or damaged by the heating process.

The opening of the housing must be arranged in such a manner that it allows sufficient access to the storage room and thus enables the cassettes to be loaded into and unloaded from the storage room with particular ease. The storage room can be designed in such a manner that it fills up the entire area or only a part thereof that is enclosed by the housing.

In a first preferred embodiment of the present invention, the gas-recirculating device comprises an inlet using which the external gas can be fed to the recirculating loop. In addition, the gas-recirculating device also comprises an outlet through which gas is discharged from the recirculating loop and can be released into the ambient air outside the housing of the heating device. The advantage of this arrangement is that the gas inside the recirculating loop is replaced by external gas and thus can be regenerated. In this connection, it is possible to exchange the gas in the recirculating loop completely or to feed external gas gradually and to continuously discharge a part of the gas mixture consisting of the original gas and the newly fed external gas out of the recirculating loop. By adjusting the quantities of gas fed and discharged from the recirculating loop, it is additionally possible to vary the gas pressure inside the recirculating loop and to thus create ambient conditions that are adapted to the respective samples.

In a second preferred embodiment of the present invention, the gas-recirculating device comprises means for processing the gas to be recirculated. The term “gas to be recirculated” refers to all the gas in the recirculating loop, that is, the gas present both in the storage room as well as in the gas-recirculating device. Using the means for processing the gas to be recirculated, it is possible to change the physical or chemical properties of the gas to be recirculated and thus to create ideal conditions for the heating process of the respective samples.

The gas-recirculating device expediently comprises a gas guide. The term “gas guide” basically refers to every medium that is suitable for guiding the gas, for instance pipelines, tubes, walls, etc. Here the gas guide is designed in such a manner that it guides the gas along the entire length of the cassette and introduces the gas into the cassette in a substantially horizontal direction for throughflow. Guiding the gas along the entire length of the cassette ensures that the gas is distributed in all the areas of the cassettes as evenly as possible, thus ensuring an even throughflow and a consequent even heating of the samples in the cassette. The gas guide is expediently arranged in such a manner that the gas is guided at least along one side of the cassette that has openings thus enabling the gas to flow through the cassette. The horizontal direction of the flow of the gas introduced into the cassette further ensures that the gas flows through all the storage areas since these are also arranged horizontally. The gas guide can be arranged both inside as well as outside the storage area. If it is arranged outside the storage area, the inlet of the gas from the gas guide into the storage room must be arranged and designed in a manner that ensures a sufficient throughflow of the gas through the cassette.

In a third preferred embodiment of the present invention, the gas guide is designed as a duct, particularly as a rectangular duct where the duct adjoins to the storage room and stretches over the length of the storage room. Since cabinet-type housings similar to the ones that are also used for the heating device in accordance with the present invention, are frequently designed as a cuboid, it is recommended to design the gas guide duct with a rectangular cross-section. The duct preferably adjoins to the storage room close to the cassette so that the gas can be guided directly onto the cassette after being introduced into the storage room from the gas guide. The cassette with its openings is expediently arranged for the feed of gas into the storage room. It is advantageous to provide the duct with a relatively simple design; for instance, it can be formed out of the walls of the housing and/or of the storage room. The fact that the gas guide duct stretches over the entire length of the storage room ensures that the gas evenly flows through the storage room and also through the cassette.

The gas-recirculating device is preferably integrated into the housing. In doing so, the gas-recirculating device can be arranged both inside as well as outside the storage room. It is also possible to arrange only one part of the gas-recirculating device inside the storage room and the other part outside it, that is, between the storage room and the housing. The advantage of this arrangement is that the integration of the gas-recirculating device into the housing enables the effective implementation of a compact design for the heating device. Alternately, the gas-recirculating device can be designed as a separate unit that can be attached to the housing using fasteners in this embodiment, preferably hinges, screws, or by welding. The advantage of this arrangement is that the gas-recirculating device can be manufactured separately from the rest of the heating device and that the final assembly must be carried out just before the initial operation. Apart from that, it is also easily possible to convert other equipment widely used in laboratories, such as for instance climatic test cabinets, refrigerators, storage devices, storage cabinets for samples, etc. and to design them as heating devices in accordance with the present invention.

A gas-recirculating device that can be attached to the housing is arranged preferably in the area of the opening of the housing and fastened to the housing by means of hinges to form a swiveling closure of the opening. Due to the attachment using hinges, the gas-recirculating device can be swung open and shut sideways just like a door, thus enabling the access to the storage room for loading and/or unloading cassettes. The gas-recirculating device expediently locks the opening of the housing tightly so as to prevent the gas from escaping from the interior of the housing.

The gas guide is preferably arranged offset to the opening of the housing. However, this is preferable only as long as the gas-recirculating device is not designed as a door for sealing the opening of the housing. This arrangement of the gas guide is advantageous because it does not adversely affect the loading and unloading of the storage room. The gas-recirculating device expediently comprises a drive unit for recirculating the gas. The drive unit is preferably designed as a blower. The blower is arranged typically in such a manner that it sucks in the gas inside the recirculating loop on one side and blows it out on the other, thus allowing for a continuous flow.

In a fourth preferred embodiment of the present invention, several cassettes are stored inside the storage room. Furthermore, the cassettes are designed to be relocatable using a means of movement and can be moved in the direction of the gas guide by means of control equipment. Due to the movement of the cassettes in the direction of the gas guide, it is possible to arrange the cassettes alternately so as to achieve a throughflow of the gas through the cassettes. The control device enables the adjustment of the movements of the cassettes, thus ensuring that the gas flows evenly through all the cassettes. Should all the cassettes contain the same samples, it is expedient to move the cassettes continuously in the direction of the gas guide so that the gas flows through every cassette for the same duration of time. In case of different samples (for instance, different contents, different temperatures, different quantities, etc.) it is possible to adjust the control equipment in such a manner that the respective cassettes dwell in the vicinity of the gas guide for different periods of time, depending on the time required by each sample for heating. In this manner, the samples can be heated with a greater degree of evenness. Moreover, the control equipment expediently controls the dwell time of the cassettes in the vicinity of the gas guide depending of the flow speed of the gas guided onto the cassettes. The dependence of the dwell time of the cassettes on the flow speed of the gas guided onto the cassettes ensures that a sufficient throughflow of the gas through the cassettes is feasible at any flow speed.

In a fifth preferred embodiment of the present invention, the means of movement comprise a horizontally swiveling or rotating cassette storage platform. The cassette storage platform is advantageously designed as a swivel plate that can rotate around its center. Generally, this embodiment is also referred to as “carousel.” Moreover, it is preferred if the means of movement comprise a transport device that is designed for transporting individual sample carriers vertically and parallel to the rotation axis of the swivel plate. The opening of the housing provides access to the storage room and the cassettes can be inserted into the storage room manually. The cassettes are arranged on the swivel plate and thus can be moved in the direction of the gas guide by rotating the swivel plate. The transport device that is already known from the U.S. Pat. No. 6,129,428 comprises a swiveling and horizontally displaceable transport element that can transport a sample carrier. The transport element is integrated into an elevator that can also move the sample carriers to be put in and out of storage. By means of an additional, sealable loading and unloading opening in the housing whose size is adjusted to that of the sample carrier, a sample carrier can be accommodated by the transport device and inserted into a storage compartment of a cassette arranged on the swivel plate. An air-locking device in the loading and unloading opening can prevent a contamination of the interior of the housing during the loading and unloading processes.

The cassettes are distributed substantially evenly on the transport platform. Consequently this improves the evenness with which the samples are heated.

Preferably the external gas is admixed to the recirculating loop using a definite ratio of mixture. This achieves a constant and continuous admixture of external gas to the recirculating loop and the gas mixture can also be adapted to suit the respective requirements of the samples.

In a sixth preferred embodiment of the present invention, the inlet of the external gas comprises a toxic substance filter and/or germ filter for filtering the external gas. The filtering of the external gas ensures that no toxic substances and germs enter into the storage room through the feed of external gas into the recirculating loop. A contamination of the samples is thus prevented effectively.

In a seventh preferred embodiment of the present invention the means for processing the gas to be recirculated comprises at least one toxic substance filter and/or germ filter through which the gas flows during the recirculation. The advantage of this arrangement is that the risk of a contamination of the samples is further reduced. The filter is preferably arranged along the gas guide. In addition, it is preferred to arrange the filter such that it is accessible from the opening of the housing. This facilitates the maintenance work and the replacement of the filters and thus generally improves the operation of the heating device.

In an eighth preferred embodiment of the present invention, the filter exhibits different thicknesses and/or different densities. These properties have a bearing on the speed at which the gas flows through the filter. In an appropriate arrangement of different levels of filtration, it is thus possible to equalize different flow speeds of the gas and to further improve the evenness of the gas flow.

In a ninth preferred embodiment of the present invention, the means for processing the gas to be recirculated additionally comprises a heater for heating the gas. Due to this it is possible to heat the gas to be recirculated at a definite temperature. Thus an ideal heating temperature for the respective samples is possible. The heater is preferably arranged along the gas guide. Here, the heater can be designed, for example, as a heating coil. The gas inside the gas guide flows past the heating coil and is thus heated up.

In addition, the heating device expediently comprises a control device that can maintain the temperature in the storage room within a definite temperature range. By this it is ensured that the samples are not exposed to any excessively high and/or excessively low temperatures and that the sample quality is not adversely affected.

The gas to be recirculated preferably consists of air. This further simplifies the structure and operation of the heating device. In this embodiment, fresh air is expediently fed to the recirculating loop as external gas. In this connection, it is particularly preferred if the proportion of fresh air in the recirculating loop amounts to approximately 10 per cent.

In a tenth preferred embodiment of the present invention, the heating device is integrated into an incubator or into a climatic test cabinet. This further simplifies the heating process of the samples since the process of rearranging the samples into a separate heating device need not be carried out any more. Apart from that, the space required inside the laboratory is also greatly reduced.

Furthermore, the objective underlying the present invention is achieved by a process for heating samples in the field of life sciences that are arranged inside a storage room and in which gas is guided onto the samples for heating the samples. In the said process, gas is recirculated in the storage room, external gas is fed to the recirculating loop, the gas to be recirculated is heated, the gas to be recirculated is filtered through germ filters and toxic substance filters and a part of the gas to be recirculated is released from the recirculating loop. These steps are repeated for the purpose of achieving a continuous heating process.

In a first preferred variant of the process in accordance with the present invention, the external gas is filtered by means of germ filters and toxic substance filters before being fed to the recirculating loop. Due to this the risk of a contamination of the samples is further reduced.

Moreover, it is preferred to feed the external gas using a predetermined ratio of mixture. Thus an ideal gas mixture can be achieved for heating the samples.

In a second preferred variant of the process in accordance with the present invention, the gas in the recirculating loop consists of air. Accordingly it is preferable if the external gas consists of fresh air. In this variant of the process, it is additionally preferable if the proportion of fresh air in the recirculating loop substantially amounts to ten per cent.

In the following description the present invention is explained more elaborately on the basis of the schematic illustrations of the embodiments of which:

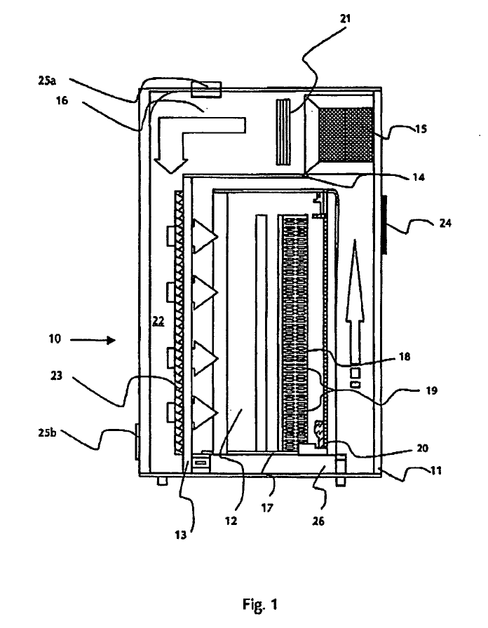

FIG. 1 illustrates a sideview of a first heating device with a gas-recirculating device integrated into the housing;

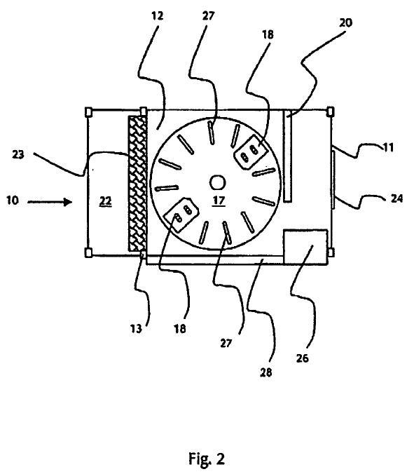

FIG. 2 illustrates the cross-section of the embodiment illustrated in FIG. 1;

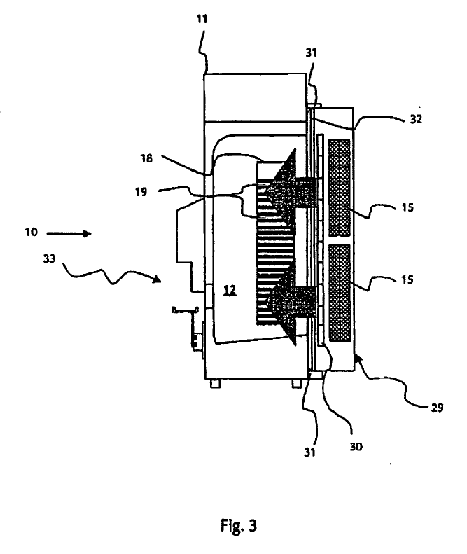

FIG. 3 illustrates a sideview of a second heating device with a gas-recirculating device attached to the housing and

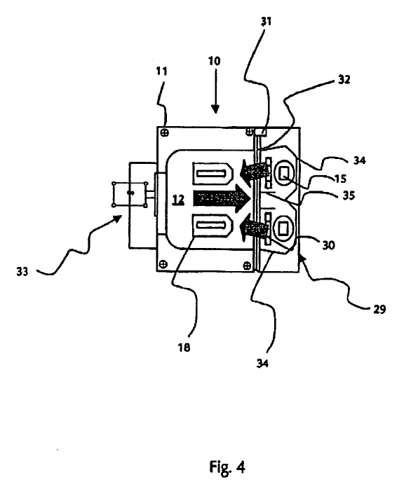

FIG. 4 the cross-section of the embodiment of the illustration illustrated in FIG. 3.

In the different embodiments illustrated in the figures, like parts have been provided with like reference symbols.

FIG. 1 illustrates a side view of a first heating device 10 with a gas-recirculating device integrated into the housing 11. In the interior of the housing 11, a storage room 12 is designed whose floor and the right and left sides of the housing are delimited by an inner wall 13. At its upper end, the storage room 12 adjoins to an intermediate ceiling 14 and a blower 15. The blower 15 sucks up the air present in the storage room 12 and blows it into a gas pipeline 16 that adjoins to the ceiling of the housing 11 and is designed as a duct. The gas pipeline 16 extends in a horizontal direction. The arrows drawn in FIG. 1 indicate the flow direction of the air and thus indicate the direction of the recirculating loop of the heating device 10.

A transport platform 17 designed as a carousel is provided inside the storage room 12 in its floor area. The circular transport platform 17 can swivel around its center. On the transport platform 17, a cassette 18 is arranged whose storage compartments 19 contain sample carriers (not illustrated here). The cassette 18 can be moved by a rotation of the transport platform 17 inside the storage room 12. The right side of the transport platform 17 adjoins to a mounting device 20 known from the patent application U.S. Pat. No. 6,129,428. The cassette 18 can be automatically equipped using the mounting device 20.

Inside the gas pipeline 16, a heater 21 is arranged directly behind the blower 15 in the flow direction. The heater 21 heats the air after it is blown from the blower 15 into the gas pipeline 16. The temperature at which the air is heated can be adjusted by regulating the heating output of the heater 21. After the air passes the heater 21, it flows again along the gas pipeline 16 in the horizontal direction till it flows into a vertically arranged gas guide 22 that is designed as a rectangular duct. The gas guide 22 is delimited on its left side by the housing 11 and on its right side by the inner wall 13. The gas guide 22 stretches over the entire length of the storage room 12 and is arranged substantially parallel to the cassette 18. This arrangement ensures that the air flowing into the gas guide 22 is distributed evenly over the entire length of the cassette 18. A toxic substance filter and/or germ filter 23 is attached to the inner side of the gas guide 22, which is delimited by the inner wall 13. In addition, inlets (not illustrated here) are arranged in the inner wall 13 through which the air flows from the gas guide 22 into the storage room 12 after having flowed through the filter 23. As can be seen in FIG. 1, the air flows approximately in a horizontal direction into the storage room 12. The cassettes 18 can be moved in the direction of the gas guide 22 by a rotation of the transport platform 17. When the cassettes 18 are close to the gas guide 22, the air flows from the gas guide 22 through the inlets (not illustrated here) through the openings of the storage compartments 19 into the cassette 18 and can thus heat up the sample (not illustrated here) present in them. The cassettes 18 usually have at least one opening and an outlet arranged on the opposite side so that the air can flow through the cassettes 18 without hindrances.

Apart from that, a gas inlet 24 is arranged on the right sidewall of the housing 11 for the feed of fresh air into the recirculating loop. The gas inlet 24 is provided with a preliminary filter (not illustrated here) through which the fresh air is filtered before it enters into the recirculating loop. A gas outlet 25a departs from the gas pipeline 16 and is arranged in the ceiling of the housing 11. The air can be released from the recirculating loop through the gas outlet 25a. Normally, the outlet 25a is adjusted in such a manner that it releases the same quantity of air that is admitted by the inlet 24 into the recirculating loop so that there is constant pressure inside the heating device 10. Alternately or additionally, the air can also be released through the outlet 25b arranged in an outer wall of the housing 11 in the lower area of the gas guide 22. The floor of the storage room 12 is delimited by a switch box 26 in which electrical components (not illustrated here) are arranged for the control and regulation of the heating device 10.

FIG. 2 illustrates the cross-section of the embodiment illustrated in FIG. 1. It must be pointed out here that the transport platform 17 comprises mountings 27 that are designed for engaging into the cassettes 18. Two cassettes 18 are arranged on the transport platform 17 opposite to one another and each of these engage into a mounting 27. The middle area of the lower sidewall of the housing 11 is designed as a door 28. The door 28 can be swung open, thus providing access to the storage room 12.

FIG. 3 illustrates a side view of a second embodiment of a heating device 10. Cassettes 18 with storage compartments 19 are arranged in the storage room 12. Samples are located in microtiter plates (not illustrated here) in the storage compartments 19. The right side of the housing 11 comprises an opening that can be sealed by the gas-recirculating device 29 attached to the housing 11. Blowers 15 and integrated heaters and filters 30 are arranged inside the gas-recirculating device 29. The gas-recirculating device 29 is swivelably attached to the housing 11 using hinges 31. The gas-recirculating device 29 is arranged in such a manner that it covers the opening of the housing 11 in its closed state. An additional layer of sealing 32 is arranged between the gas-recirculating device 29 and the housing 11. On the left sidewall of the housing 11, an automatic mounting device 33 is attached using which the cassettes 18 can be equipped automatically with sample carriers. Furthermore, it must be pointed out that the recirculated air flows from the gas-recirculating device 29 approximately horizontally and into the storage room 12 and also through the storage compartments 19 of the cassettes 18 in the horizontal direction.

FIG. 4 illustrates the cross-section of the embodiment illustrated in FIG. 3. It must be pointed out here that inside the gas-recirculating device 29, an area separated by inner walls 34 is present in which the blowers 15 and the integrated heater/filters 30 are arranged. The sides of this area separated by the inner walls 34 end flush with the storage area 12. Two additional internal partition walls 35 are arranged inside this area. The inner walls 34 and the internal partition walls 35 are designed to be continuous from the floor up to the ceiling. The internal partition walls 35 are arranged parallel to the sidewalls of the housing 11 and start at the internal end of the gas-recirculating device 29 and end in the open space. Thus the inner area of the gas-recirculating device 29 is sub-divided into three sub-areas, all of which are connected to one another and can exchange air among one another. The arrows indicate the flow direction of the air. Air is sucked in by the blowers 15 and subsequently blown out again in the direction of the storage room 12. In doing so, it first flows through the integrated heater/filter device 30 and then flows further through inlets (not illustrated here) in the sealing layer 32 into the storage room 12. Both the cassettes 18 are each arranged close to an inlet. Air flows through both the cassettes in the horizontal direction. The air flows through the cassettes 18, collects in the rear area of the storage room 12, and then flows centrally through a gap between the two cassettes 18 and back into the gas-recirculating device 29.

1. Heating device for samples from the field of life sciences having at least one cassette with horizontally arranged storage compartments for storing sample carriers, in which gas is supplied to the cassette for heating the samples comprising:
a housing with a storage room that is designed for the feed and discharge of gas and for the storage of at least one cassette and;
a gas-recirculating device for recirculating the gas in the storage room.

2. Heating device pursuant to claim 1, wherein the gas-recirculating device comprises an inlet for the admixture of external gas into the recirculating loop as well as an outlet for the discharge of a part of the gas to be recirculated out of the recirculating loop.

3. Heating device pursuant to claim 1, wherein the gas-recirculating loop comprises means for processing the gas to be recirculated.

4. Heating device pursuant to claim 1, wherein the gas-recirculating device comprises a gas guide that guides the gas along the entire length of the cassette and introduces the gas into the cassette in a substantially horizontal direction.

5. Heating device pursuant to claim 4, wherein the gas guide is designed as a duct that adjoins to the storage room and stretches over the length of the storage room.

6. Heating device pursuant claim 1, wherein the gas-recirculating device is integrated into the housing.

7. Heating device pursuant to claim 1, wherein the gas-recirculating device can be placed on the housing and attached to it using fasteners, particularly hinges, screws, bolts or by welding.

8. Heating device pursuant to claim 7, wherein the gas-recirculating device is attached to the housing by means of hinges in the area of the opening of the housing and is thus designed as a swiveling closure of the opening.

9. Heating device pursuant to claim 4, wherein the gas guide is arranged offset to the opening of the housing.

10. Heating device pursuant to claim 1, wherein the gas-recirculating device comprises a drive unit, particularly a blower, for recirculating the gas of the storage room.

11. Heating device pursuant to claim 1, wherein the heating device comprises several cassettes that are relocatable using means of movement and can be moved in the direction of the gas guide by using control equipment.

12. Heating device pursuant to claim 11, wherein the control equipment controls the speed and/or the frequency of the displacement of the cassettes depending on the flow speed of the gas guided onto the cassettes.

13. Heating device pursuant to claim 11, wherein the means of movement comprise at least one horizontally swiveling or rotating cassette storage platform.

14. Heating device pursuant to claim 13, wherein the cassette storage platform is designed as a swivel plate.

15. Heating device pursuant to claim 13, wherein the means of movement comprise a transport device for automatically inserting the sample carriers into and removing them out of the storage room where the transport device for displacing the sample carriers is arranged vertically and parallel to the rotation axis of the cassette storage platform of the cassette.

16. Heating device pursuant to claim 13, wherein the cassettes are substantially evenly distributed on the cassette storage platform.

17. Heating device pursuant to claim 2, wherein the external gas is admixed to the ambient gas of the storage room using a predetermined ratio of mixture.

18. Heating device pursuant to claim 2, wherein the inlet for the external gas comprises a toxic substance filter and/or germ filter in order to filter the external gas.

19. Heating device pursuant to claim 3, wherein the means for processing the gas to be recirculated comprises at least one toxic substance filter and/or germ filter through which the gas flows during the recirculation process.

20. Heating device pursuant to claim 19, wherein the filter is arranged along the gas guide.

21. Heating device pursuant to claim 19, wherein the filter is designed with many thicknesses and/or different densities.

22. Heating device pursuant to claim 19, wherein the filter is arranged such that it is accessible from the opening of the housing.

23. Heating device pursuant to claim 3, wherein the means for processing the gas to be recirculated comprise a heater for heating the gas during the recirculation process.

24. Heating device pursuant to claim 23, wherein the heating device is arranged along the gas guide.

25. Heating device pursuant to claim 1, wherein a control device is provided by means of which it is possible to maintain the temperature inside the storage room below and/or above a predetermined temperature.

26. Heating device pursuant to claim 1, wherein the gas to be recirculated consists of air.

27. Heating device pursuant to claim 1, wherein the heating device is integrated into an incubator or a climatic test cabinet.

28. Process for heating samples in the field of life sciences that are arranged inside a storage room and in which gas is guided onto the samples for heating them; comprising the following steps:
a) recirculating the gas in the storage room;
b) feeding external gas to the recirculating loop
c) heating the gas to be recirculated;
d) filtering the gas to be recirculated using germ filters and/or toxic substance filters;
e) discharging a part of the gas to be recirculated out of the recirculating loop and
f) repeating the steps a) to e).

29. Process pursuant to claim 28, wherein the external gas is filtered before being fed to the recirculating loop in the same way as in step d).

30. Process pursuant to claim 28, wherein the external gas is fed using predetermined ratios of mixture.

31. Process pursuant to claim 28, wherein the ambient gas of the storage room consists of air.

32. Process pursuant to claim 31, wherein the external gas consists of fresh air.

33. Process pursuant to claim 32, wherein the proportion of fresh air in the recirculating loop substantially amounts to 10%.
